(19) United States
(12) Patent Application Publication
(10) Pub. No.: US 2010/0286485 A1
(43) Pub. Date: Nov. 11, 2010

(54) Adaptable tissue retractor with plurality of movable blades

(76) Inventors: Valerio Valentini, Montreal (CA); Anthony Paolitto, Montreal (CA)

Correspondence Address: Anthony PAOLITTO;CORONEO INC., Suite 514, 9250 Avenue du Parc, Montreal, QC, H2N 1Z2, CA

(21) Appl. No.: 12/662,696

(22) Filed: Apr. 29, 2010

Related U.S. Application Data
(60) Provisional application No. 61/213,075, filed on May 5, 2009.

(51) Int. Cl. A61B 1/32

(52) U.S. Cl. 600/224

(57) ABSTRACT

An adjustable multi-bladed tissue retractor capable of producing an incrementally variable or fine-tunable retracted opening in a surgical incision, or a body cavity. The adjustable tissue retractor comprises an actuator coupled to a retractor housing and a movable linkage arrangement capable of moving the plurality of tissue-engaging blades attached thereto, between a closed-blade configuration and an open-blade configuration. Through the application of a predetermined input to the actuator, a controlled, or selectable spaced apart spatial relationship of the plurality of tissue retracting blades results, and whereby, in use, the tissue retractor may be customized, or individually tailored to suit the specific anatomy of the patient.

This application claims the benefits of U.S. Provisional Patent Application 61/213,075 filed May 5, 2009.

### FIELD OF THE INVENTION

The present invention relates to the field of surgical instruments to retract a body tissue and more specifically, to adaptable tissue retractors that may be adjusted in use to comply with a specific anatomy of a patient, or specific geometry of a surgical incision, body cavity or opening.

### BACKGROUND OF THE INVENTION

Current tissue retractors, especially in cardiac surgery, are typically of a fixed geometry. They are most commonly configured at the tissue-retracting end with either a “basket” type configuration made from spaced apart wire frame members, or with an uninterrupted and shaped tissue contacting surface or blade that engages the body tissue to be retracted. Consequently, the tissue-retracting end of these existing retractors is not adjustable or adaptable to suite the specific anatomy of the patient, but it is the patient's anatomy that is urged to adapt to the fixed geometry configuration of such existing tissue retractors. Tissue retractors with malleable tissue-retracting ends may be manually bent by the user, prior to engaging same with the body tissue to be retracted, but such manipulation is not selectively fine-tunable a predetermined amount while the tissue retractor is deployed in use, and the tissue-retracting end thereof is in contact with the body tissue being retracted. Usually, such malleable tissue retractors may only be grossly configured to approximate the most desirable configuration required. Increasing the malleability of the tissue-retracting end may result in insufficient retractor stiffness to be able to positively retract the body tissue under load.

In cardiac surgery requiring the retraction of heart tissue, for instance in a mitral valve surgery practiced via a left atrial approach, commonly used retractor platforms include the “Cosgrove-type” and “Carpentier-type” retractor platforms. In using the “Cosgrove-type” retractor platform (see FIG. 1), generally three fixed geometry basket-type tissue retractors are deployed to retract the incised cardiac tissue. Each of these tissue retractors is independently mounted or secured to a sternal retractor, or stable surgical platform, to achieve the desired retraction of the atrial incision and to obtain surgical access to the target mitral valve. In using the “Carpentier-type” retractor platform (see FIG. 2), generally two tissue retractors with fixed shape tissue contacting surface are deployed and mounted to a sternal retractor. The lack of adaptability with these current tissue retractors, and their inability to spread or retract a body tissue in more than one traction direction, results in the need to deploy a plurality of tissue retractors each independently mounted to the surgical platform generally in a different traction direction. Consequently, the associated surgical set-up is time-consuming given the high part count, and the ergonomics of the surgical worksite is compromised given the plurality of individually-mounted tissue retractors to the sternal retractor.

In-process re-adjustments of the plurality of tissue retractors with known valve surgery platforms may at times prove fastidious. For example, in a typical set-up with a Cosgrove-type retractor platform, two tissue retractors are generally mounted on a left sternal retractor spreader arm to retract cardiac tissue towards the patient's left side. A third tissue retractor is mounted on an extension rod or member to retract cardiac tissue towards the patient's feet. The extension rod is also mounted to the left sternal retractor spreader arm in a substantially perpendicular and generally horizontal orientation. If the surgeon wants to modify the orientation of the tissue-engaging or tissue-retracting end of the middle tissue retractor, for instance, the mounting clamp of the middle tissue retractor must be repositioned along the left sternal retractor spreader arm. Larger re-orientations generally require more translation of the mounting clamp along the left sternal retractor spreader arm. In certain cases, the re-orientation required is sufficiently great that the mounting clamp of the middle tissue retractor must be translated considerably along the left sternal retractor arm that it may interfere with the mounting clamp of an adjacent tissue retractor. This may lead to a major take town of the surgical set-up.

Recently, with the advent of minimally invasive cardiac surgery gaining in popularity, the size of the surgical access incision and the size of the retracted surgical access thoracic opening are being progressively reduced. Vacuum-assisted venous drainage has been developed to reduce the size of venous cannulae used in cardiopulmonary bypass. The smaller sizes of these cannulae tend to prevent them from being obstructive to the surgical procedure. However, the number of traction sutures and number of tissue retractors, either hand held or chest-retractor-mounted, currently used in existing approaches tends to be obstructive in certain areas given the relatively smaller size of the surgical window.

### SUMMARY OF THE INVENTION

Thus, it is a first object of the present invention to provide a single, solitary tissue retractor configured with a plurality of tissue engaging blades (four or five blades), said tissue retractor being adaptable or adjustable in the relative positioning of the blades, and whereby, in use, the tissue retractor may be customized, or individually tailored to suit the specific anatomy of the patient or the specific geometry of a surgical incision with a desired spatial relationship of the plurality of blades.

It is a further object of the present invention to be able to mount one such multi-bladed tissue retractor to a stable surgical platform and obtain the proper access to the target tissue to be operated, said target tissue being located within the perimeter of the retracted surgical incision or body cavity, without the need to deploy multiple individually bladed tissue retractors, each individually or each independently mounted to a stable surgical platform.

It is a further object of the present invention to be able to retract tissue in a primary direction, and simultaneously by deploying blades from a blade closed to a blade open configuration, retract in a second retraction direction being substantially at (+90) degrees relative to this first primary direction, and also in a third retraction direction being substantially at (−90) degrees relative to this first primary direction and generally diametrically opposite to the second retraction direction.

It is another object of the present invention to provide an adjustable multi-bladed tissue retractor capable of producing an incrementally variable or fine-tunable retracted opening or retraction perimeter through the application of a predetermined input to an actuator on the tissue retractor that results in a controlled, spaced apart spatial relationship of the plurality of tissue-retracting blades.

It is another object of the present invention to provide an adjustable tissue retractor configurable or adaptable to achieve a desired tissue retraction with a plurality of blades that may be selectively interspaced between a closed blade configuration and an open blade configuration, said open blade configuration resulting in a substantially circular or arcuate retraction span of 200+/−20 degrees (with 4 blades) or 320+/−40 degrees (with 5 blades).

These and other objects of the present invention will become apparent from the description of the present invention and its preferred embodiments which follows.

### BRIEF DESCRIPTION OF THE DRAWINGS

For better understanding of the present invention and to show more clearly how it may be carried into effect, reference will now be made by way of illustration and not of limitation to the accompanying drawings, which show a tissue retractor apparatus according to preferred embodiments of the present invention, and in which:

FIG. 1 is a perspective view of a prior art surgical platform commonly known as a “Cosgrove-type” retractor platform;

FIG. 2 is a perspective view of a prior art surgical platform commonly known as a “Carpentier-type” retractor platform;

FIG. 3 is a perspective view of a multi-bladed tissue retractor being configured with four tissue-retracting blades according to a preferred embodiment of the present invention;

FIG. 4 is a top view of a multi-bladed tissue retractor illustrated in FIG. 3, with the tissue-retracting blades being in a closed-blade configuration;

FIG. 5 is a top view of a multi-bladed tissue retractor illustrated in FIG. 3, with the tissue-retracting blades being deployed in an open-blade configuration;

FIG. 6 is an enlarged bottom view of the tissue retractor in FIG. 3 according to a preferred embodiment of the present invention;

FIG. 7 is an exploded view of the tissue retractor illustrated in FIG. 3 according to a preferred embodiment of the present invention;

FIG. 8 is a cross-sectional view of a portion of the tissue retractor illustrated in FIG. 3 according to a preferred embodiment of the present invention;

FIG. 9 is a top view of a multi-bladed tissue retractor having five tissue-retracting blades according to a second embodiment of the present invention.

### DETAILED DESCRIPTION OF THE INVENTION

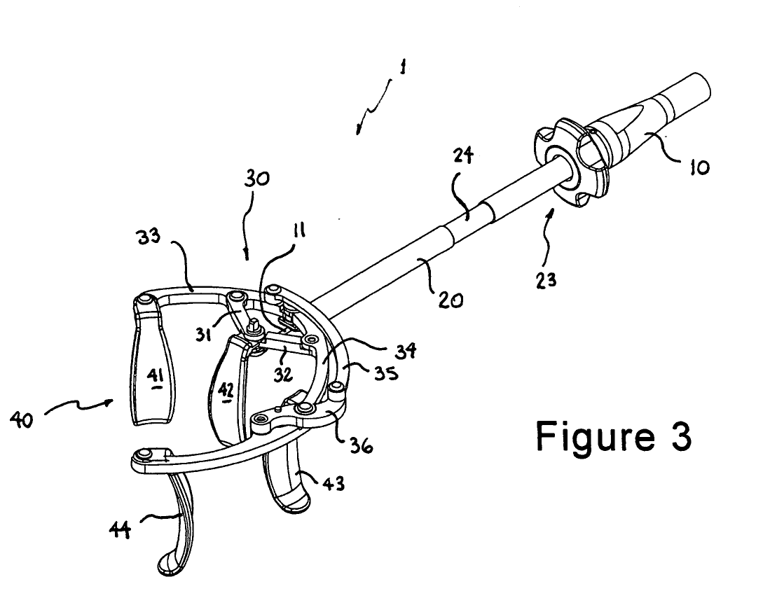

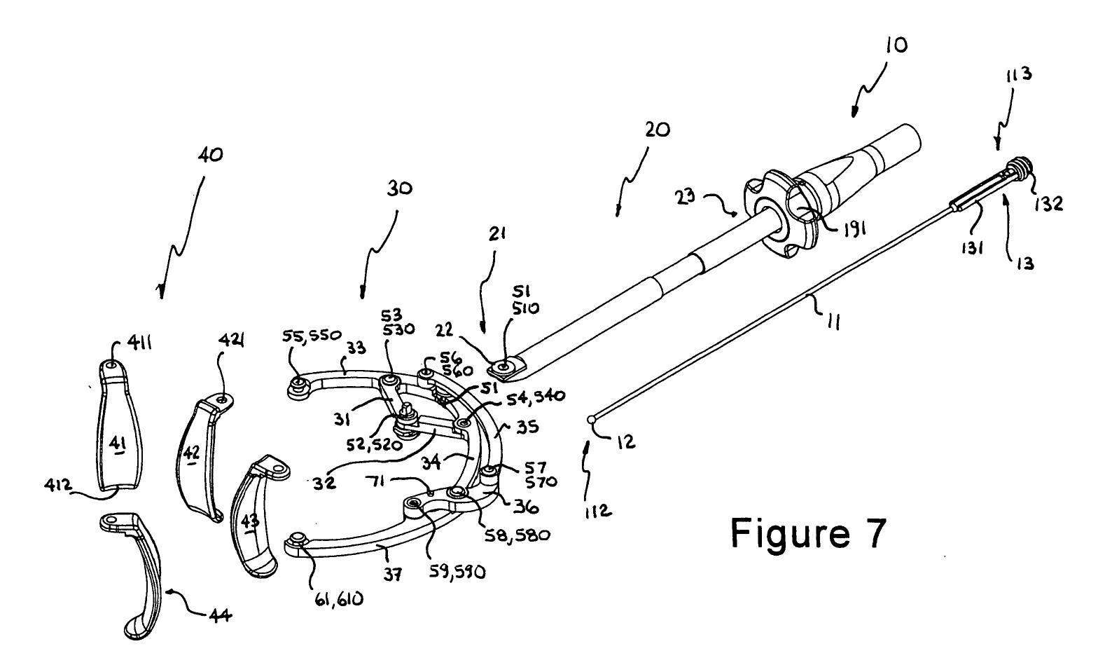

With reference to FIGS. 3 and 7, tissue retractor 1 is comprised of an actuator or actuating means 10, a tubular housing 20, a linkage mechanism, assembly or arrangement 30, and a plurality of tissue-contacting, tissue-engaging or tissue-retracting blades, fingers or members 40. Linkage arrangement 30 is mechanically coupled to tubular housing 20 at a first or distal end 21 thereof, through mechanical joint 22.

Linkage assembly 30, as a whole, is pivotingly engaged and able to pivot relative to housing 20 about pivot axis 510, regardless of the blade configuration assumed by tissue-engaging blades 40 and by virtue of flexible cable 11 as will be described in greater detail below. Linkage assembly 30 is able to articulate in a multitude of different linkage configurations, and consequently able to transmit a multitude of blade 40 spatial geometries, relative to said housing. As such, tissue retractor 1 is adaptable or adjustable to the desired retraction geometry or configuration.

Actuator 10 is mechanically coupled to tubular housing 20, at a second or proximal end 23 thereof. More specifically, and with reference to FIG. 8, actuator 10 is preferably rotatingly engaged to said housing proximal end 23 via a rolling element bearing, such as ball bearing 28. End 23 of housing 20 is configured with an external circumferential groove 281 adequately sized to act as a bearing inner race for said bearing 28. Actuator 10 consists of a first, knob part 19 configured with lobes 191 and assembled through a threaded interface 185 to a second, cooperating tubular part 18. Knob part 19 is provided with a circumferential inner groove 192 and tubular part 18 with a second inner groove 182. Grooves 192, 182 cooperate to form a circumferential outer race for bearing 28 when said parts 18, 19 are assembled together via threaded interface 185, thereby axially retaining the rolling elements of bearing 28 within said grooves and also axially retaining said actuator 10 relative to said housing 20. The trapped bearing now allows the free, low friction rotation of actuator 10 relative to housing 20.

Actuator tubular part or member 18 extends beyond terminal end of housing 20 to expose an internal thread 15. Housing 20 is elongate and extends between first 21 and second 23 housing ends. Housing 20 is provided with an internal passageway or through bore 26 that extends between said first 21 and second 23 ends of tubular housing thus providing open communication therebetween. Said passageway is configured and sized to house or receive an actuating member therewithin, in the nature of a translating actuating cable 11. Cable 11 is preferably flexible, and fabricated from surgical grade braided stainless steel wire. Cable first end 112 is provided with a spherical or ball end 12 configured to be insertable into a receiving socket or depression 301 disposed on linkage assembly 30 so as to allow for a demountable mechanical interface between actuator cable 11 and said linkage assembly, said mechanical interface secured in its mounted state through latch or clasp member 302. Cable second proximal end 113 is provided with a threaded member or fitting 13 configured with an external thread portion 132 sized to engage internal thread 15 in actuator part 18 when said cable 11 is inserted in housing passageway 26. Fitting 13 is also provided with an elongated tongue or key member 131 which is configured and sized to engage a longitudinal slot, groove or keyway 27 in terminal end of housing 20. Keyway 27 communicates passageway 26 with the outer surface of tubular housing 20 and extends longitudinally along housing longitudinal axis at least as long as the length of tongue 131. When cable 11 is inserted into passageway 26, tongue 131 is first slidingly engaged with keyway 27, and subsequently external thread 132 engages internal thread 15 in actuator 10.

During actuation of actuator 10, applying an actuation input in the nature of a rotation to knob 19 results in a translation of cable 11 through housing 20 since tongue 131 and groove 27 cooperate to prevent said cable from rotating together with knob 19 as internal thread 15 urges or entrains cable external thread 132 to translate axially along with cable 11 relative to housing 20. The resulting translation of actuation cable 11 relative to tubular housing 20 results in the extension (or retraction) of said cable beyond said housing distal end 21, which in turn simultaneously entrains the articulation of linkage mechanism 30, as will be described in greater detail below.

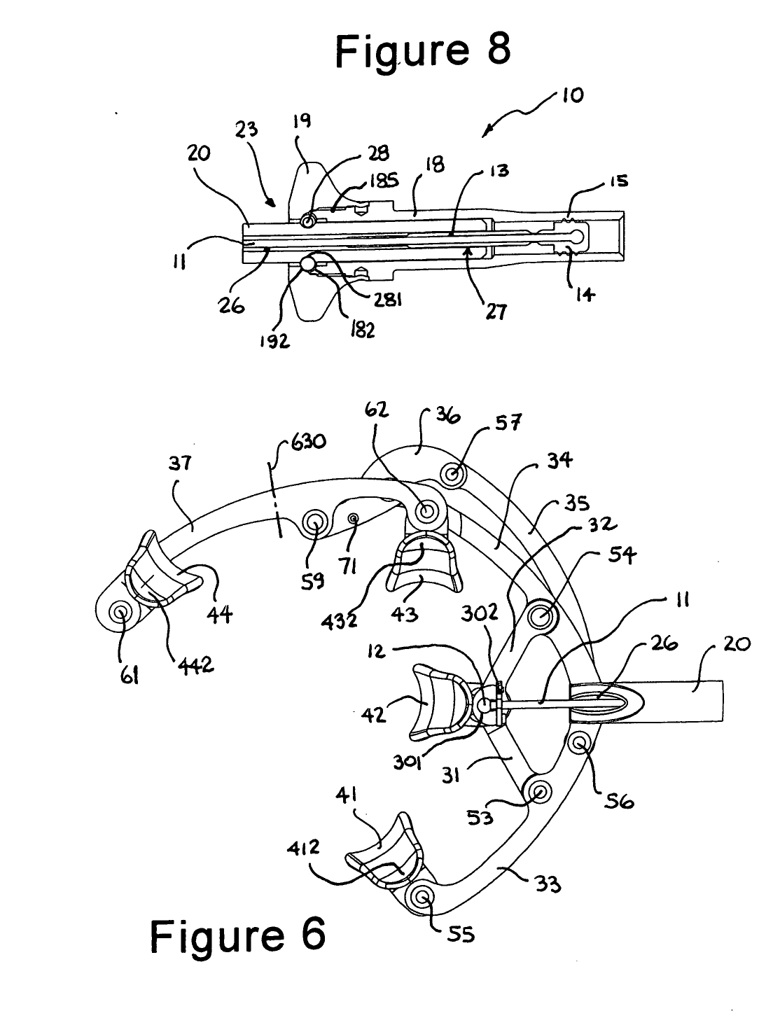

As illustrated in FIG. 6, the center of socket 301, that demountably engages terminal ball end 12 of actuator cable 11, is generally aligned with pivot axis 520 of mechanical joint 52. Joint 52 is configured to pivotingly engage linkage members 31 and 32 to each other, as well as blade 42 which is also pivotingly engaged thereto via its through-hole 421. Linkage member 33 is pivotingly engaged, at a first end, to housing 20 through mechanical joint 51, and it pivots about linkage pivot axis 510. Blade 41 is pivotingly engaged to linkage member 33 at a blade mount location in the nature of mechanical joint 55, and it pivots about blade pivot axis 550. Approximately at the mid length location of linkage member 33, linkage members 31 and 33 are pivotingly engaged through mechanical joint 53. Linkage member 31 pivots relative to linkage member 33 about linkage pivot axis 530. Linkage member 34 is pivotingly engaged, at a first end, to housing 20 through mechanical joint 51, and it pivots about linkage pivot axis 510. Approximately at the mid length location of linkage member 34, linkage members 32 and 34 are pivotingly engaged through mechanical joint 54. Linkage member 33 pivots relative to linkage member 34 about linkage pivot axis 540. At a second end, linkage member 34 is pivotingly engaged to cross linkage member 36 at mechanical joint 58 located generally at the mid-span of member 36. Cross linkage member 36 is pivotingly engaged to blade carrier linkage member 37 through mechanical joint 59. Linkage member 37 is able to pivot relative to cross member 36 about pivot axis 590, said pivot range being limited by mechanical stop feature or pin 71 extending proudly from surface of cross member 36 to limit the pivoting range of carrier member 37 relative to member 36. Blades 43 and 44 are each pivotingly coupled to a respective free end of linkage member 37 via blade mounts or mechanical joints 62 and 61, respectively. Said blades 43, 44 are able to pivot about blade pivot axes 620 and 610, respectively. The pivoting range of cross member 36, relative to linkage member 34, is set by coupling linkage member 35 which is pivotingly engaged to a second terminal end of cross member 36 at mechanical joint 57, and pivotingly engaged to linkage member 33 at mechanical joint 56. Coupling linkage member 35 pivots relative to member 33, about pivot axis 560. Cross member 36 pivots relative to member 35 about pivot axis 570.

When actuator 10 is actuated by applying a rotational actuation input 100, the extended portion of actuating cable 11 (FIG. 6) is progressively retracted into passageway 26 of housing 10. This moves blade 42 generally along a first movement direction “Y” towards tubular housing end 21. Said translation of blade 42 entrains linkage members 31 and 32 to pivot in generally opposed directions relative to mechanical joint 52 where the latter are coupled, and simultaneously linkage members 33 and 34 to also pivot in generally opposed directions relative to housing 20 where the latter are coupled or connected at mechanical joint 51 on said housing. Consequently, blade 41 moves or swings in an arcuate trajectory about pivot axis 51, said trajectory corresponding to movement or a change in position along both “X” and “Y” directions. Similarly, mechanical joint 58 moves in a generally opposed arcuate trajectory to blade 41, also about pivot axis 51. The resulting differential change in position along “X” and “Y” of mechanical joint end 57 relative to mechanical joint 58 for a given translation of cable 11 (and blade 42) along direction “Y”, establishes the amount that cross linkage member 36 pivots about axis 580, and consequently also the position along “X” and “Y” of mechanical joint 59.

Pivot joint 59 acts as a fulcrum for carrier linkage member 37 which carries spaced apart blades 44, 43 from each other and from a fulcrum coincident with pivot axis 59. This spacing between the fulcrum 59 and each of the respective blade pivot axes 610, 620 may be designed such that the orientation assumed by said carrier linkage 37 relative to the rest of linkage mechanism 30 will be determined by the relative force exerted on each of blades 43, 44 by the body tissue being retracted, said latter forces being magnified as a function of the moment arm (or spacing) between fulcrum 59 and respective blade pivot axes 610, 620 to reach equilibrium of the force couple exerted on blades 43, 44. As such, the position of blades 43, 44 is set along direction “X” and “Y” as a function of the magnitude of translation of cable 11 along direction “Y”. The spacing between fulcrum 59 and each of respective blade pivot axes 610, 620 may be advantageously designed according to the type of incision or body cavity being retracted.

Additionally, mechanical stops or restraints 71 may also be included between cross linkage 36 and carrier linkage 37 to intentionally limit the range of articulation of one linkage member relative to the other, and override the equilibrium orientation that would otherwise be achieved without the implementation of said mechanical stops 71. Similar mechanical stops may also be incorporated between blades 41, 42, 43, and 44 and the respective linkage member pivoting coupled to said blades to limit the pivoting range of said blades about their respective pivot axes 550, 520, 620 and 610.

Cable 11 is preferably flexible so as to allow flexing of the exposed cable portion extending beyond housing 20. When blades 41, 42, 43, and 44 are engaged with a body tissue to be retracted, this provides adaptability by allowing the entire linkage mechanism 30 to articulate and reorient itself as an entire assembly relative to tubular housing 20, in any given linkage configuration (i.e blade closed, blade open, or intermediately therebetween). In addition, the adaptability of each of blades 41, 42, 43, and 44 to pivot and orient themselves optimally relative to the body tissue being retracted results in a less traumatic tissue retraction. This adaptability tends to provide substantially equal or equilibrated reaction loads being applied by each blade to the contacted portion of body tissue being retracted.

Blades 41, 42, 43, and 44 are configured and sized for a particular tissue to be retracted. For instance, as illustrated in FIG. 3, blades 40 are intended to retract heart tissue such as tissue of the left atrium through a left atrial incision. Accordingly, terminal blade ends 412, 422, 432, and 442 are bent to act as hook or retracting members during left atrial tissue retraction, but are also profiled to be blunt and atraumatic so as to not pierce body tissue.

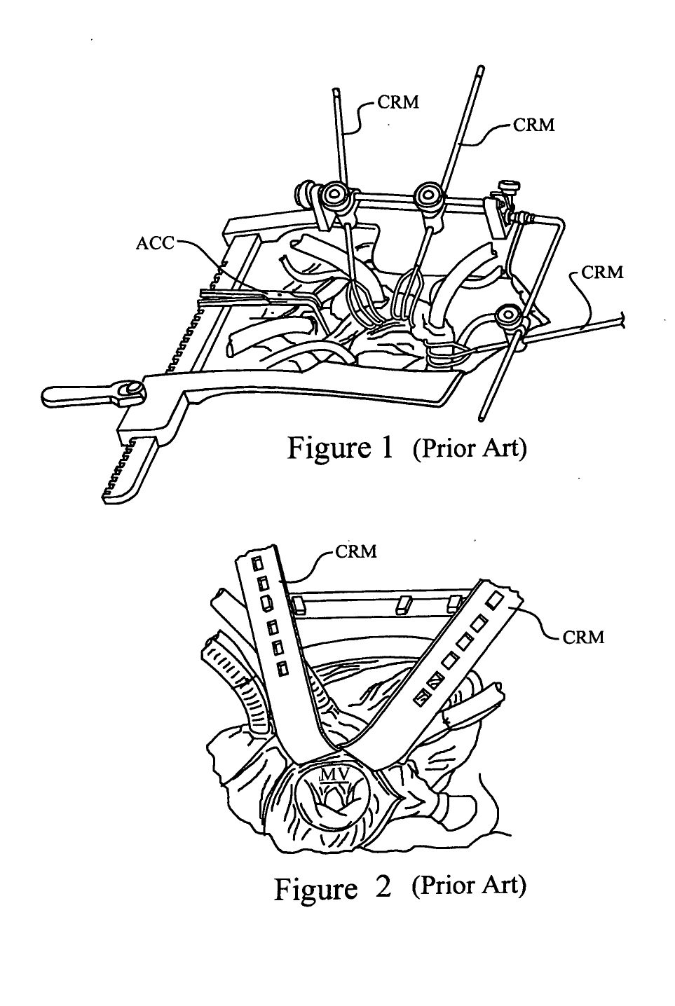

During deployment of tissue retractor 1, said actuator 10 may rotate relative to said housing 20 in order to effect or apply an actuation input 100 to said tissue retractor which will deploy, adjust, or adapt the plurality of tissue-contacting blades 40 into a desired spatial arrangement suitable for a surgical procedure. Incremental variations in the actuation input 100 will result in a similar incremental variation in said spatial arrangement of tissue-engaging blades 40. As such, a surgeon or user of said tissue retractor 1 may apply a predetermined actuation input to said actuator 10 to achieve a desired deployment or adjustment of said tissue-engaging blades 40, said spatial relationship of blades 40 being well suited for the retraction of a particular surgical incision, or the opening of an anatomical body cavity. Tubular housing 20 is advantageously provided with a seat 24 for mounting or engaging said tissue retractor 1 to a substantially stable surgical platform such as, for instance, a sternal retractor for cardiac surgery as described in U.S. Pat. No. 6,837,851. Mounting tissue retractor 1 to a sternal retractor for cardiac surgery as the one recited in U.S. Pat. No. 6,837,851 will tend to avoid the hereinabove described drawbacks associated with individually mounting each of a plurality of known fixed geometry, basket-type tissue retractors to a “Cosgrove-type” retractor platform, as illustrated in FIG. 1.

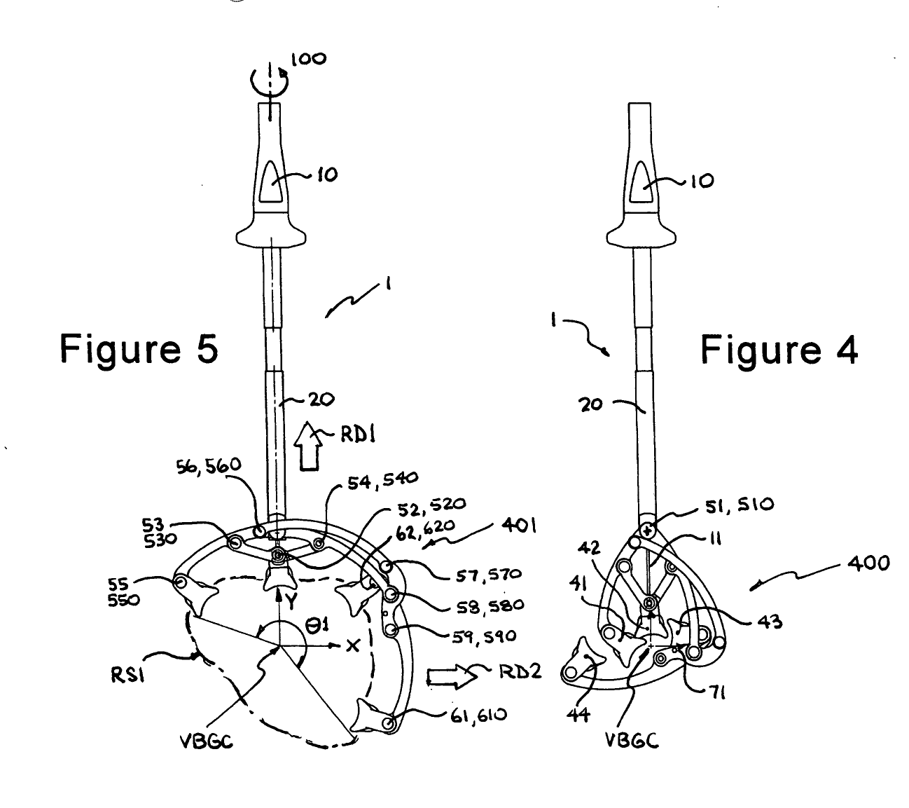

We will now describe in greater detail the deployment configurations of tissue retractor 1. In a closed-blade configuration 400 as illustrated in FIG. 4, tissue-retracting blades 40 are in a substantially compact, closely-spaced blade arrangement and are disposed generally about a virtual blade geometric center “VBGC”. Said closed-blade configuration is advantageous to facilitate insertion of said tissue retractor 1, and more specifically blades 40 thereof, in a non-retracted surgical incision or facilitate placement in a non-retracted body cavity. Then, as tissue retractor 1 is deployed by actuator 10, said tissue retractor assumes a plurality of incrementally variable blade spatial positions whereby the relative spacing between blades is adjustable. The degree of blade opening is “fine-tunable” or selectable, and may be optimally set as a result of the actuation input 100 applied by the user to actuator 10, so as to configure the adaptable tissue retractor to best adapt to the specific incision, or specific anatomy of the patient.

With reference to FIG. 5, tissue retractor 1 is illustrated in an open-blade configuration 401, said open-blade configuration being set to retract a surgical incision of body cavity schematically depicted as “RSI”. The span of tissue retraction established by four spaced apart blades 41, 42, 43, and 44 in said open-blade configuration 401 forms an arc of retraction Θ1 approximately 200+/−20 degrees, disposed generally around or about VBGC of blade 40 arrangement. As illustrated in FIG. 5, said open-blade arrangement (and more specifically blades 41, 42, and 43) may provide retraction along a first retraction direction RD1 when said tissue retractor 1 is manipulated or displaced to apply a retraction load to RSI along a direction generally aligned with longitudinal axis of housing 20. As well, blade 44 may also provide a simultaneous retraction along a second direction RD2 , said RD2 direction being offset at 90 degrees relative to RD1. In the context of a cardiac surgery, and more specifically, valve surgery performed on a mitral valve via left atrial approach, tissue retractor 1 may be deployed so that blades 41, 42, 43 retract a first portion of a left atrial incision upwardly along RD1 so as to rotate and lift heart within patients thorax, while blade 44 moves laterally to said blades 41, 42, 43 to retract a second portion of said left atrial incision along RD2 generally towards patient's abdomen to improve surgical access to diseased mitral valve. In cases where a shorter left atrial incision is made, the pivoting range of blade 44 about pivot axis 610 allows said blade 44 to reorient itself through a clockwise rotation. As such, blade 44 will impart a retraction load having at least a force component that pulls or retracts tissue downwardly (i.e. in the negative RD1 direction), or at a generally opposed incision side relative to blades 41, 42, 43 which cooperate to support the bulk of the weight of the patient's heart.

Those skilled in the art of linkage mechanisms will appreciate that offset between pivot axes 560 and 510 as well as the offset between pivot axes 570 and 580 may be varied in the design of tissue retractor 1 in order to optimize a rate of displacement of blade 44 relative to other blades 41, 42 (or relative to the center of the retracted opening or VBGC). For instance, linkage mechanism 30 may be designed so that blade 44 moves laterally away from center of retracted opening or VBGC at a faster rate at the beginning of range of open-blade positions relative to the end of range of open-blade positions, for a given fixed rate of translation of action cable 11 relative to housing 20.

Alternatively, carrier linkage member 37 may be configured with an additional pivot joint (shown schematically as dashed line 630 in FIG. 6) so as to allow blade 44 to pivot into or out of main retracting plane X-Y (plane of page of FIGS. 5 and 6), for instance +/−20 degrees out of plane X-Y. This provides yet another additional degree of adaptability to tissue retractor 1 to allow said tissue retractor to more optimally adapt to certain surgical incision geometries or specific patient's anatomies with the aim of achieving less traumatic tissue retraction.

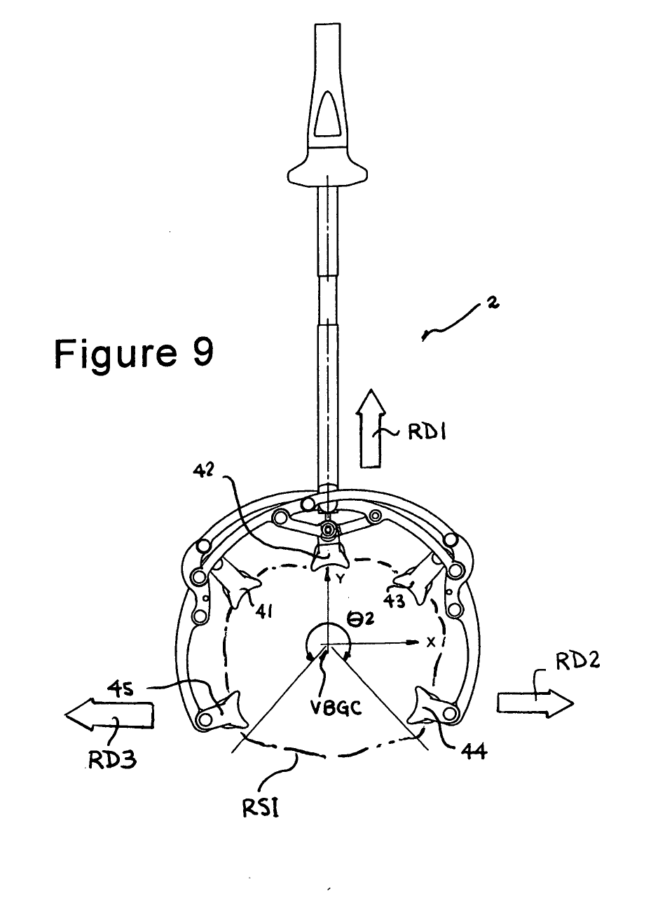

FIG. 9 illustrates a second embodiment of a tissue retractor 2 according to the present invention. Tissue retractor 2 includes five tissue-retracting blades 41, 42, 43, 44, and 45. The span of tissue retraction established by said five spaced apart blades in an open-blade configuration 402 forms an arc of retraction Θ2 approximately 320+/−40 degrees, disposed generally around or about VBGC of blade 41, 42, 43, 44, 45 arrangement. As illustrated, said open-blade arrangement may provide retraction along a first retraction direction RD1 (blade 42, component of blade 41, component of blade 43) when said tissue retractor 1 is manipulated or displaced to apply a retraction load to RSI along a direction generally aligned with longitudinal axis of housing 20. As well, blade 44 may also provide a simultaneous retraction along a second direction RD2, said RD2 direction being offset at 90 degrees clockwise relative to RD1. As well, blade 45 may also provide a simultaneous retraction along a third direction RD3, said RD3 direction being offset at 90 degrees counterclockwise relative to RD1, or being generally in opposed direction to RD2. In certain types of surgical incisions or body cavity retractions, typically those smaller in size, tissue retractor 2 may be deployed so that the arc of retraction Θ2 substantially approximates a complete circumferential span, said span being retracted by five spaced apart tissue-retracting blades.

1. A tissue retractor for retracting a body tissue, said tissue retractor comprising:
four tissue-engaging blades, said tissue-engaging blades configured and sized for retracting the body tissue, said tissue-engaging blades each connected to a blade mount location of a movable linkage mechanism, said linkage mechanism coupled to an actuator, said tissue retractor movable between a closed-blade configuration and an open-blade configuration by the actuation of said actuator, wherein in said closed-blade configuration said four tissue-engaging blades are in proximity to each other, and in said open-blade configuration said four tissue-engaging blades are in a spaced apart spatial relationship, said spaced apart spatial relationship being variably selectable by the degree of actuation input applied to said actuator.

2. A tissue retractor according to claim 1, wherein said linkage mechanism includes a plurality of linkage members, each of said linkage members being pivotingly coupled to at least one other linkage member in said linkage mechanism.

3. A tissue retractor according to claim 2, further comprising a housing, said linkage mechanism coupled to said housing and coupled to said actuator via an actuating member, said housing configured and sized to house said actuating member, said actuating member simultaneously coupled to said actuator and at least to one of said linkage members, whereby when said actuator is actuated, said actuating member moves relative to said housing and entrains the movement of said linkage mechanism so as to move said tissue-engaging blades between said closed-blade and open-blade configuration.

4. A tissue retractor according to claim 3, wherein said linkage mechanism is pivotingly connected to said housing through at least one of said linkage members, and wherein said actuator is actuated by applying a rotation to said actuator relative to said housing, said applied rotation resulting in a translation of said actuating member relative to said housing, said actuating member translation resulting in a pivoting of said at least one linkage member pivotingly connected to said housing, said pivoting of said at least one linkage member entraining the movement of interconnected plurality of pivotingly-engaged linkage members and the simultaneous movement of said tissue-engaging blades between said closed-blade and open-blade configuration.

5. A tissue retractor according to claim 1, further comprising a fifth tissue-engaging blade, said fifth tissue-engaging blade configured and sized for retracting the body tissue, said fifth tissue-engaging blade connected to a fifth blade mount location of said movable linkage mechanism wherein in said closed-blade configuration said five tissue-engaging blades are in proximity to each other, and in said open-blade configuration said five tissue-engaging blades are in a spaced apart spatial relationship, said spaced apart spatial relationship being variably selectable by the degree of actuation input applied to said actuator.

6. A tissue retractor for retracting a body tissue, said tissue retractor comprising:
four tissue-engaging blades, said tissue-engaging blades configured and sized for retracting the body tissue, said tissue-engaging blades coupled to an actuator, said tissue retractor movable between a closed-blade configuration and an open-blade configuration by the actuation of said actuator, wherein in said closed-blade configuration said four tissue-engaging blades are in proximity to each other to forma a substantially compact blade arrangement, said compact blade arrangement defining a virtual blade geometric center, and in said open-blade configuration said four tissue-engaging blades are in a spaced apart spatial relationship, said spaced apart spatial relationship being variably selectable by the degree of actuation input applied to said actuator, said spaced apart spatial relationship spanning to form an arc of tissue retraction disposed generally about said virtual blade geometric center.

7. A tissue retractor according to claim 6, wherein in said open-blade configuration, said arc of tissue retraction resulting from the spacing apart of said four tissue-engaging blades is approximately 200+/−20 degrees.

8. A tissue retractor according to claim 6, further comprising a fifth tissue-engaging blade, said fifth tissue-engaging blade configured and sized for retracting the body tissue, said fifth tissue-engaging blade coupled to said actuator wherein in said closed-blade configuration said five tissue-engaging blades are in proximity to each other, and in said open-blade configuration said five tissue-engaging blades are in a spaced apart spatial relationship, said spaced apart spatial relationship being variably selectable by the degree of actuation input applied to said actuator, said spaced apart spatial relationship spanning to form an arc of tissue retraction disposed generally about said virtual blade geometric center.

9. A tissue retractor according to claim 8, wherein in said open-blade configuration, said arc of tissue retraction resulting from the spacing apart of said five tissue-engaging blades is approximately 320+/−40 degrees.
